(19) United States
(12) Patent Application Publication
(10) Pub. No.: US 2011/0295199 A1
(43) Pub. Date: Dec. 1, 2011

(54) PLANNING AND ASSEMBLY OF COMPENSATING CONCENTRIC CANNULAS

(75) Inventors: Aleksandra Popovic, New York, NY (US); Karen Irene Trovato, Putnam Valley, NY (US)

(73) Assignee: KONINKLIJKE PHILIPS ELECTRONICS N.V., EINDHOVEN (NL)

(21) Appl. No.: 13/142,456

(22) PCT Filed: Nov. 10, 2009
(86) PCT No.: PCT/IB2009/054995

§ 371 Date: Jun. 28, 2011

Related U.S. Application Data
(60) Provisional application No. 61/141,130, filed on Dec. 29, 2008.

(51) Int. Cl. A61M 25/092; G06G 7/48; G06G 7/60

(52) U.S. Cl. 604/95.01; 703/11

(57) ABSTRACT

A specification for a device including a set of concentric cannulas may be discovered to have an actual path different from a desired path, due to interactions between cannulas that effect net curvature of the device. The choice of particular cannulas may be corrected by performing a calculation taking into account curvature affecting properties of the individual cannulas including radius of curvature, elasticity, and moment of inertia. This calculation is preferably performed iteratively starting with a most distal cannula and iterating through the cannulas to the proximal end, accumulating net effect of curvature affecting properties.

The invention relates to the field of planning and construction of telescoping concentric cannulas for insertion into a patient.

The following related applications and patent documents are incorporated herein by reference:
U.S. Pat. No. 4,949,277, issued Aug. 14, 1990 to Trovato et al.
U.S. Pat. No. 5,879,303, issued Mar. 9, 1999 to Averkiou et al.
U.S. Pat. No. 6,604,005, issued Aug. 5, 2003 to Dorst et al.
Prior, U.S. application Ser. No. 12/088,870 of Trovato et al., filed Oct. 6, 2006 (3D Path Planning, Simulation and Control System), U.S. Patent Application Publication no. 2008/0234700, Sep. 25, 2008.
Prior, U.S. provisional applications no.'s 61/075,886, Jun. 26, 2008 and 61/099,223, Sep. 23, 2008, of Trovato et al. (Method and System for Fast, Precise Path Planning), which is International application no. PCT/IB2009/052650, filed Jun. 19, 2009.
Prior, U.S. provisional application No. 61/106,287 of Greenblatt et al., filed Oct. 17, 2008 (Interlocking Nested Cannula), which is International application no. PCT/IB2009/054474, filed Oct. 12, 2009.
Prior, International application no. IB2007/053253 of Trovato, filed Aug. 15, 2007 (Active Cannula Configuration for Minimally Invasive Surgery), International Publication no. WO 2008/032230 A1, Mar. 20, 2008.
Prior, U.S. Provisional application No. 61/075,401 of Trovato, filed Jun. 25, 2008 (Nested Cannulae for Minimally Invasive Surgery)), which is International application no. PCT/IB2009/052521, filed Jun. 12, 2009.

These documents, when taken cumulatively, describe a medical application, which will be roughly summarized as follows:

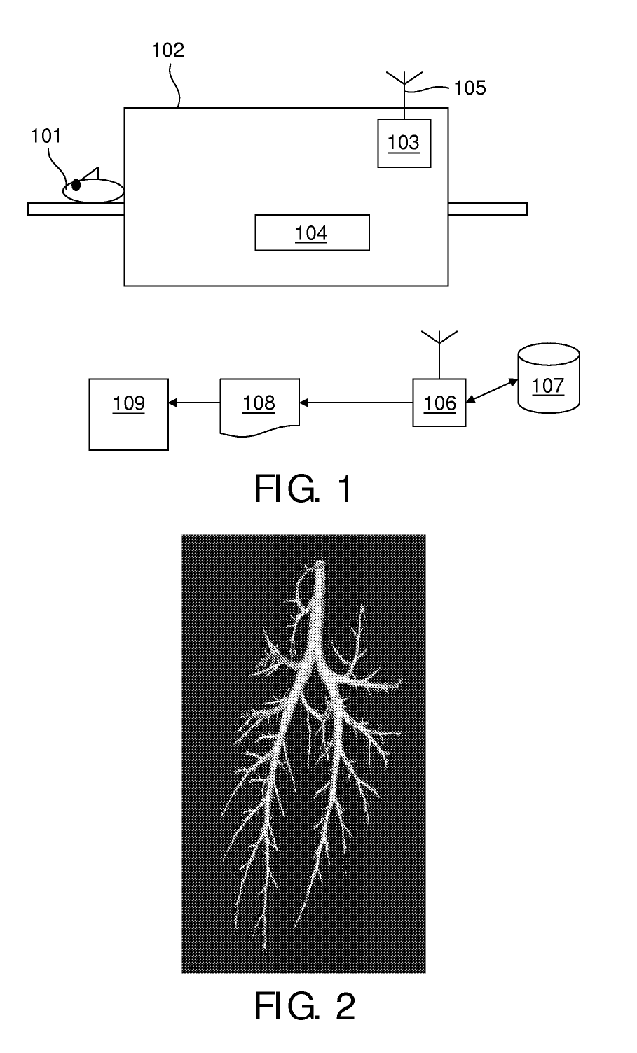

In FIG. 1, a patient 101 is scanned in a scanning device 102. The scanning device may be of any suitable type, such as ultrasound, CT scanning, or MRI scanning. Any portion of the patient's body may be scanned, for example the lungs. The result of the scan will be to show interior structure of the patient's body. The interior structure may include tubular passages, such as the airways of a lung, blood vessels, the urethra, nasal passages or intestines. The interior spaces may be more open, such as the stomach, the bladder or the sinuses. In some cases the interior structure will be solid tissue, but be where certain areas are preferred, for instance within the brain. The medical application is not limited to any particular scanning technique or any particular interior space of the body.

The scanning device will include a processor 103 for gathering and processing data from the scan. The processor may be of any suitable type and will typically include at least one machine readable medium for storing executable program code and data. There may be multiple processors and multiple storage media of one or more different types. The processor will often have some way of communicating with outside devices. This processor is illustrated with an antenna 105 for wireless communication, but the communication might equally well be wired such as to the Internet, infrared, via optical fiber, or via any suitable method. The scanning device will also include at least one user interface 104, including one or more of: a display, a touch sensitive screen, a keyboard, a pointer device, a microphone, a loudspeaker, a printer, and/or any other user interface peripheral. The invention is not limited to any particular peripherals for communicating with a user or with outside equipment.

While all processing may occur within the scanning device, there may also be an outside processor 106 for performing planning of a path, and an assumed set of ‘net shapes’ to follow the path. The processor 106 will be associated with at least one medium 107 for storing data and program code. The medium 107 may include various types of drives such as magnetic, optical, or electronic, and also memory such as cache where executing code and data structures may reside. The output of the planning process is illustrated schematically and includes a technical specification 108 in any appropriate format and also the concentric cannulas 109 themselves.

FIG. 2 shows an image of tubular passages in a patient's lungs segmented from a scan. It is desirable to insert medical devices into the tubular passages, since this minimizes damage en route to a target location. This type of surgery is called NOTES (Natural orifice translumenal endoscopic surgery) when an endoscope is used to travel through passages. This type of surgery does not require that the surgical target be within the tubular access, but rather that the target is reached with less trauma by having tools that travel through existing tubes, so that the target may be reached translumenally.

Tubular devices, such as Active Cannulas, have been proposed, see e.g. R. J. Webster et al., “Toward Active Cannulas: Miniature Snake-like Surgical Robots” 2006 IEEE/RSJ (October 2006, Beijing, China) pp. 2857-2863. These devices rely on the interaction between two or more tubes to cause lateral motion as they rotate relative to one another. As they extend from one another, they can also cause various lateral motions, particularly if they have different curvatures along a single tube. If the motion is carefully characterized, these motions can be used to reach multiple locations, similar to a robot in free space. However these devices can have difficulty when extended translumenally, if the lateral motion is greater than the available maneuver space. While the Webster article considers interactions of tubes during deployment, it lacks consideration of issues relating to making Active Cannulas follow a planned path.

Such devices may assist in gathering data, gathering tissue, or performing other procedures. Based on a patient image, for example, a set of tubes can be extended, from largest to smallest so that, when deployed, they have a structure where at least a portion of each cannula will remain at the proximal end of the patient while smaller cannulas will extend into the patient interior space in reverse order of diameter. Thus the fattest cannulas will end more proximally, while the thinnest cannulas will extend more distally. Herein a cannula will be considered more distal if it ends more distally when deployed—and more proximal if it ends more proximally when deployed.

Nested Cannulas are somewhat different from Active Cannulas, since they are configured to reach specific locations in a specific environment with minimal lateral motion (wiggle). In one variety of Nested Cannula, the tubes are interlocked so that they do not rotate with respect to one another. Insertion should minimize trauma to the tubular passageways or other tissues. Such trauma can result from movements of the cannulas. Nested Cannulas are described in, inter alia, the related application U.S. provisional No. 61/106,287, filed Oct. 17, 2008, set forth above.

FIG. 3 shows schematically an example of the process to be followed. First, the patient is scanned at 301. An image is then created at 302 indicating forbidden regions and, typically, the costs for passing through other regions. For example, the image may be segmented to extract the airways from the rest of the image as shown in FIG. 2. Then a path is planned including a series of shapes at 303. As described in prior path planning applications, this requires defining a seed location to start the search. Subsequently, a concentric cannula device is built to achieve the specified shapes, which is received by the practitioner at 304. Finally, a desired procedure may be performed on the patient at 305 by extending the tubes in the order specified.

Given the flexibility of modern technology, many of these operations may be performed remotely. For instance, data may be processed into a model of the interior space (e.g. segmented) in one location. A path through the space and a device suitable for following that path may be planned in a second location. Then the device may be assembled in a third location, before being returned to the technician or physician for insertion into the patient. Preferably, assembly of the nested cannula device will be performed in a manufacturing facility with good quality and sanitary controls; nevertheless, it might be that all these steps could be performed in a single location with the physician herself assembling the device to be inserted.

It has been proposed to use A* style path planning to facilitate deployment of active cannulas, see e.g. “3D Tool Path Planning, Simulation and Control System,” U.S. Ser. No. 12/088,870, filed Oct. 6, 2006, U.S. Patent Application Publication no. 2008/0234700, Sep. 25, 2008, which is incorporated in its entirely by reference herein and made a part of this application. This type of planning makes use of a “configuration space.” A “configuration space” is a data structure stored on at least one machine readable medium. The configuration space represents information about a physical task space. In this case, the physical task space is the interior structure of the patient's body into which the active cannulas are to be inserted. The configuration space includes many “nodes” or “states,” each representing a configuration of the device during insertion.

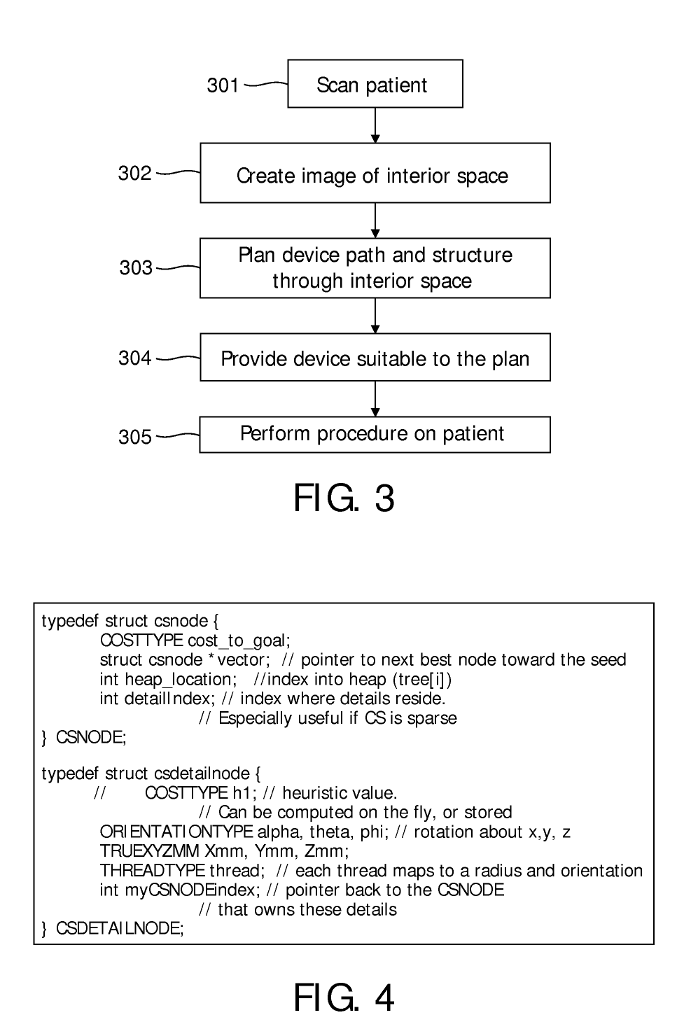

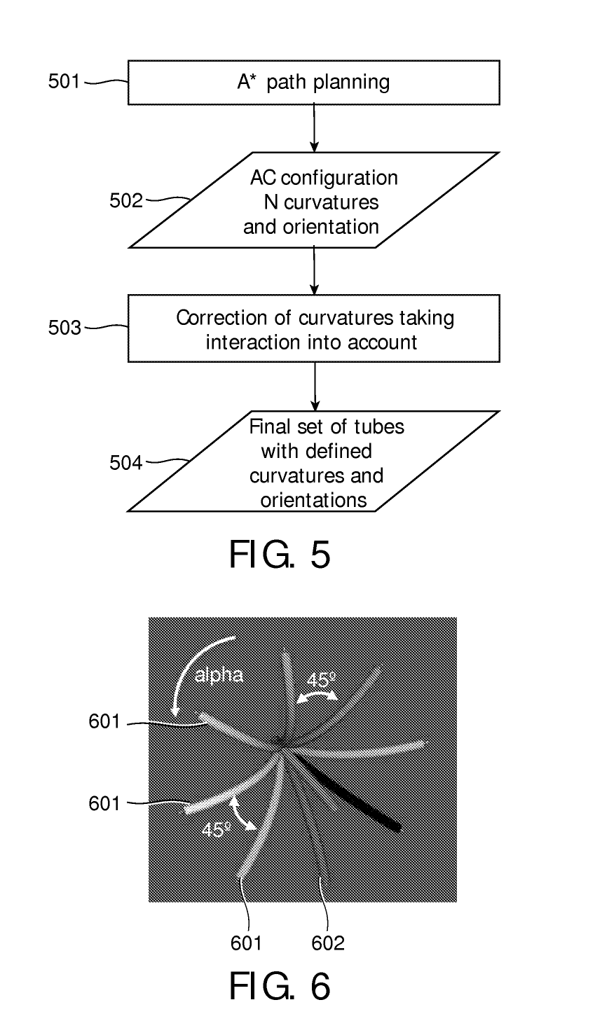

FIG. 4 shows source program code for creating a node in a configuration space as taught by U.S. Provisional Application No. 61/075,886 of Trovato et al., preferably improved to minimize memory using the method taught in U.S. Provisional Application 61/075,886 of Trovato et al. Such program code is converted to machine executable code and embodied on a medium for use by the invention. When the code is executed, it will give rise to the configuration space data structure as embodied on a medium. This particular code has been found to be advantageous with respect to interior spaces of the human body. This code allows a 6D space to be compressed into 3D, by augmenting 3D configuration space paths, with high precision locations and orientations rather than inferring them from their configuration state position.

A* or ‘cost wave propagation,’ when applied to the configuration space, will search the configuration space, leaving directions, such as a pointer, leading to the ‘best path to the seed’ at every visited state. “Propagation of cost waves” involves starting from a search seed, often a target point. Propagation of cost waves through the configuration space data structure makes use of an additional type of data structure embodied on a medium known as a “neighborhood.” The neighborhood is a machine-readable representation of permissible transitions from one state in the configuration space to other states within the configuration space. For example in FIG. 6, a single curvature of a single arc (also called a fiber) is shown at eight evenly spaced rotations relative to a given location. The lengths of the arcs might be limited to less than 90 or 180 degrees depending upon the application, and the thread shown in the center (zero curvature arc) might also be limited to approximately the same length.

Propagation of cost waves also involves a “metric,” which is a function that evaluates the cost incurred due to transitioning from one state to a neighboring state.

The term “concentric cannulas” will be used herein to include Active Cannulas and Nested Cannulas, as described above. The present invention is applicable to both types.

An advantageous material for use in Active Cannulas is Ni—Ti alloy (nitinol). Nitinol has “memory shape”, i.e. the shape of a nitinol tube/wire can be programmed or preset at high temperatures. Therefore, at lower temperatures (e.g. room or body temperature) if a smaller tube extends from a larger one, it returns to its ‘programmed shape’. Another advantage of nitinol is that it can be used within an MRI machine. It is a relatively strong material and therefore can be made thin walled, enabling the nesting of several tubes. Tubes with an outer diameter from 5 mm down to 0.2 mm of 0.8 mm and below are readily available in the market. Other materials, such as polycarbonate may also be used, particularly for low cost, interlocking Nested Cannulas.

The result of planning is preferably
a deployable physical set of concentric cannulas; and/or
a specification of the set of cannulas in terms of number, length, and radius of bending.

Certain areas for improvement remain with respect to the existing method and apparatus. For instance, trauma to patient tissues could be reduced by adjusting the specification of the set of concentric cannulas after it is planned, taking into account expected interactions of the tubes responsive to curvature affecting properties of the tubes. Such curvature affecting properties include radius of curvature, elasticity modulus, and moment of inertia.

Further objects and advantages will be apparent in the following.

The following figures illustrate the invention by way of non-limiting example.

FIG. 1 shows a patient being scanned.

FIG. 2 shows an example segmentation of a lung indicating the airways.

FIG. 3 is a schematic flow diagram of the process in which the invention is to operate.

FIG. 4 shows an example of program code for a configuration space state (CSNODE).

FIG. 5 is a flowchart showing post planning elasticity corrections.

FIG. 6 shows a schematic of an example of a neighborhood of a type having eight threads, each representing a possible path choice based on possible tube choices.

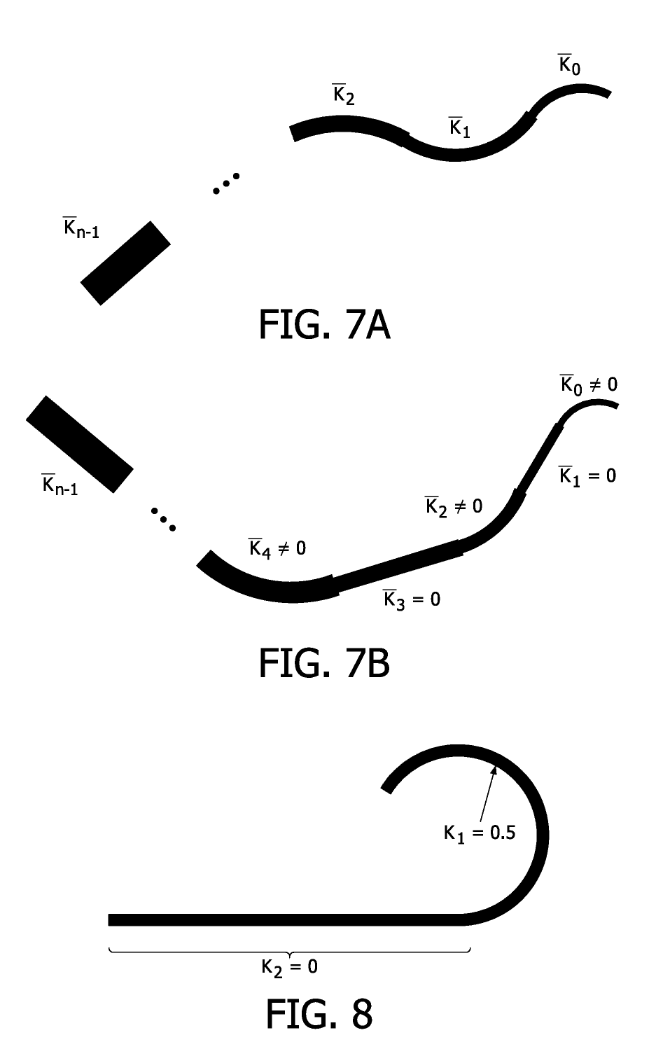

FIG. 7A is a picture of cannulas with various radii of curvature.

FIG. 7B shows an assembly of concentric cannulas with alternating curved and straight segments.

FIG. 8 shows a tube with more than one radius of curvature.

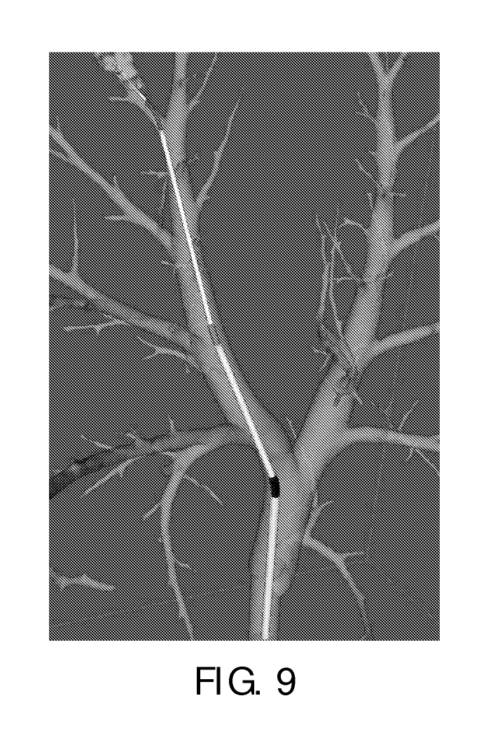

FIG. 9 shows an assembly of concentric cannulas deployed within a lung.

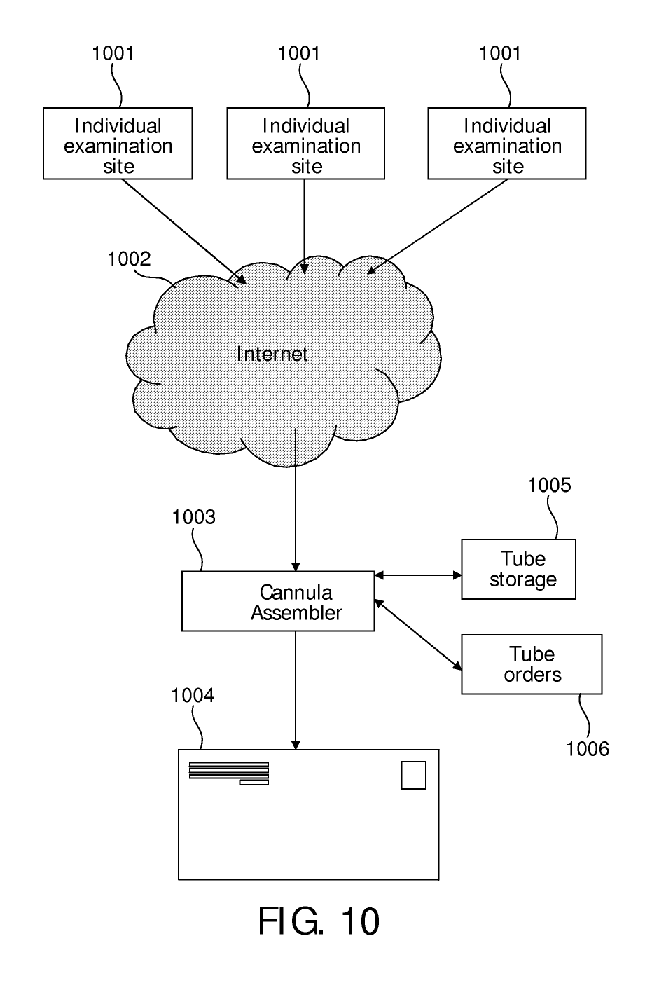

FIG. 10 is a schematic diagram of the ordering and manufacturing process relating to assemblies of concentric cannulas.

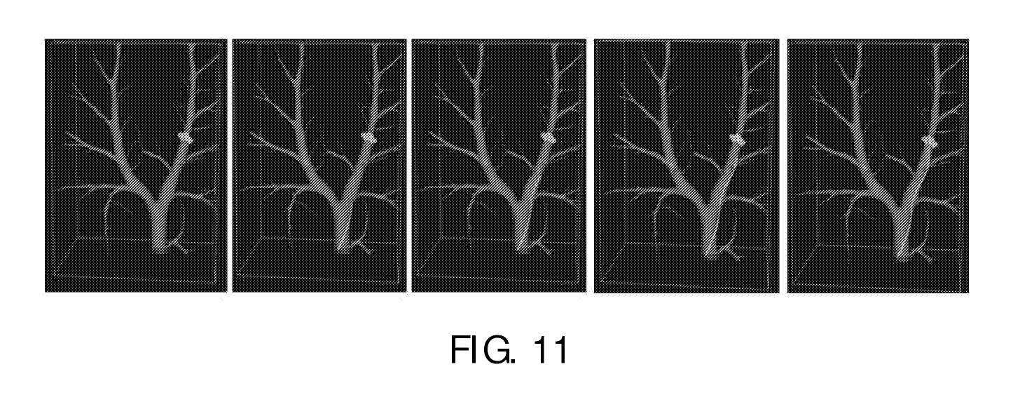

FIG. 11 is an animation relating to specification of an assembly of concentric cannulas.

Herein, the terms “tube” and “cannula” will be used interchangeably to refer to components of the device to be deployed. The phrases “radius of curvature,” “radius of bending,” and “tube curvature” will all be used interchangeably to refer to the curvature of a tube. The terms “radius,” “diameter,” “tube radius,” and “tube diameter” will all be used to refer to geometrical dimensions of a cross section of a tube. “Net curvature” will be used to refer to the curvature of an assembly of tubes resulting from individual properties of each component tube.

The fields of applicability of the invention are envisioned to include many types of procedures including imaging, chemotherapy, chemoembolization, radiation seeds, photodynamic therapy, neurosurgery, laparoscopy, vascular surgery, and cardiac surgery. It is also possible that concentric cannula in accordance with the invention could be used for non-medical applications where there are difficult to reach spaces, perhaps at the interior of a machine to be repaired.

A model of how cannulas interact mechanically with each other is to be found in the Webster et al. article cited below in the Bibliography. From this article, it can be seen that concentric cannulas will have curvatures and elasticities that are a result of combined effects of all the cannulas in areas of overlap. As Active Cannulas rotate with respect to each other, both their joint curvature and curvature plane change. Therefore, the cannulas perform two movements: tip movement and lateral movement of the device. Whereas tip advancement is a desired feature, lateral movement of component cannulas of the device might cause a collision with tissue, possibly causing damage.

One approach to creating concentric cannula devices is to consider tube interaction during planning. More about such a model is discussed in a co-pending application applicants' docket no. 011868, filed concurrently herewith.

Another approach is to define the shapes of the component cannulas based on a path, post hoc. This requires performing calculations relating to tube interaction after the path is determined. A procedure for doing this is shown in FIG. 5.

At 501, path planning occurs, for instance per U.S. application Ser. No. 12/088,870 of Trovato et al.

The result, at 502, is a concentric cannula configuration, e.g. a path including n alternating straight and arc segments. This configuration may take the form of n sets of { κ̅ᵢ, α̅ᵢ, Iᵢ}, where κ̅ᵢ are curvatures of segments, α̅ᵢ are angular orientations of the segments, and Iᵢ is the moment of inertia of the tube cross-section. Values { κ̅ᵢ, α̅ᵢ} represent net curvatures and net orientations resulting from interactions of assembled tubes. Values {κᵢ,αᵢ} represent curvatures and orientation of tubes before being assembled.

FIG. 6, while illustrating a neighborhood for path planning, may also be thought of as illustrating some possible angular orientations of the curved segments, e.g. at 601, along with a straight tube 602 in the center. In FIG. 6, the angle alpha αᵢ is shown as being measured counterclockwise in the plane of the figure. The discretization chosen is for eight different angles, a symmetric set, with 45° between adjacent curves. The skilled artisan might choose more or less angles as required for a desired level of precision based on the specific application, or depending upon the manufactured tubes—e.g. six evenly spaced threads would match a hexagonally shaped tube for use in a Nested Cannula device. More angles provide more options during planning, but may increase computation time. The skilled artisan must balance these factors to choose the discretization. The angular orientation αᵢ is defined for each tube. Frequently the angles are evenly distributed, however this is not required. For example, the alpha values may not include the tube opposite the current orientation, so as to reduce situations that may maximally stress the tubes.

At 503, a calculation is performed correcting the deployment plan in view of elastic interaction between the cannulas.

### Interaction Between Two Tubes

The absolute value of the curvature of a tube in a plane is defined as the reciprocal value of the bending radius. The “curvature vector” is oriented perpendicular to the bending plane.

Interaction between n tubes (or wires) with the same angular rotation is defined as follows:

$${\overline{κ}}_{r} = \frac{{\sum }_{i = 0}^{n - 1} {F}_{i} {κ}_{i}}{{\sum }_{i = 0}^{n - 1} {F}_{i}} , \qquad ( 1 )$$

where κ̅ᵣ is resulting curvature in the plane and κᵢ, i=0 . . . n−1 are curvatures of the interacting tubes. Fᵢ are tube specific parameters, i.e.:

Fᵢ=Eᵢ·Iᵢ, (2)

where Eᵢ is elasticity modulus (i.e. Young's modulus) of the i-th tube and Iᵢ is the moment of inertia of the cross section of i-th tube.

To simplify calculation herein, it will be assumed that all the tubes are made of the exactly same material, so that Eᵢ=E, ∀i and Iᵢ=const₁·(rₒ⁴−rᵢ⁴), where rₒ⁴ and rᵢ⁴, are outer and inner radius of the tube, respectively, and const1 is a constant number, with

$${const}_{1} = \frac{π}{64} .$$

The skilled artisan might alter the device to include different materials. In such a case, the calculation would have to be altered to reflect that. Hence:

Fᵢ=E·Iᵢ=const·(rₒ⁴−rᵢ⁴). (3)

Where const is a constant numer, const=const₁*E. If the curvatures of the tubes are angularly rotated with respect to each other, the angular interaction has to be considered, and the resulting curvature has two planar components. The generalized form of the elastic interaction between two tubes is given as:

$${\overline{\vec{κ}}}_{r} = \frac{1}{{E}_{1} {I}_{1} + {E}_{2} {I}_{2}} \cdot [ \begin{matrix}{E}_{1} {I}_{1} \cdot {κ}_{1} \cdot sin {α}_{1} + {E}_{2} {I}_{2} \cdot {κ}_{2} \cdot sin {α}_{2} \\ {E}_{1} {I}_{1} \cdot {κ}_{1} \cdot cos {α}_{1} + {E}_{2} {I}_{2} \cdot {κ}_{2} \cdot cos {α}_{2}\end{matrix} ] \qquad ( 4 )$$

Angles α₁ and α₂ are rotation angles around a reference axis. The resulting curvature vector ({right arrow over ( κ̅ᵣ)) is a 2D vector:

$${\overline{\vec{κ}}}_{r} = [ \begin{matrix}{\overline{κ}}_{r} \cdot sin \overline{{α}_{r}} \\ {\overline{κ}}_{r} \cdot cos \overline{{α}_{r}}\end{matrix} ] , \qquad ( 5 )$$

where K̅ᵣ is the absolute value of the resulting curvature and āᵣ is the resulting angular rotation of the tube in the same coordinate system as in Eq. (4).

### More Tubes

The more general case is to compute physical curvatures and angles of tubes (κᵢ i=0 . . . n−1 and αᵢ i=0 . . . n−1). Once the device is deployed, the physical curvature of the smallest and therefore most distal tube will be unaffected by other tubes, since it will extend alone. This most distal tube is designated as the “zero” tube and becomes an input to the model in the form:

κ₀= κ̅₀ and α₀= α̅₀

meaning that for the “zero” tube, net curvature and net angle are equal to physical curvature and physical angle. During deployment, there will typically be an interaction between more than two tubes, e.g. three tubes with moments Iᵢ, Iᵢ₊₁, Iᵢ₊₂, curvatures κᵢ, κᵢ₊₁, κᵢ₊₂ and angles αᵢ, αᵢ₊₁, αᵢ₊₂ starting with outermost tube. To simplify computation, the resulting curvature will be computed using Eq. (4) using the fact that two nested tubes (e.g. i and i+1), if interacting with a third one act as one tube, having the moment of inertia I₁=Iᵢ+I₊₁, and curvature κ₁= κ̅ᵢ. The third tube acts as one tube, defining I₂=Iᵢ₊₂ and κ₂=κᵢ₊₂.

### Model Computation

Equation (4) can be rewritten in the following form:

$$[ \begin{matrix}\overline{κ} \cdot sin \overline{α} \\ \overline{κ} \cdot cos \overline{α}\end{matrix} ] = \frac{1}{{I}_{1} + {I}_{2}} \cdot [ \begin{matrix}{I}_{1} \cdot {κ}_{1} \cdot sin {α}_{1} + {I}_{2} \cdot {κ}_{2} \cdot sin {α}_{2} \\ {I}_{1} \cdot {κ}_{1} \cdot cos {α}_{1} + {I}_{2} \cdot {κ}_{2} \cdot cos {α}_{2}\end{matrix} ] \qquad ( 6 )$$

Given that initial values (κ₀ and α₀) are known, the problem reduces to interactively solving n−1 sets of two equations (Eq. (6)) with two values unknown (κ₂ and α₂) and given that E can be canceled out. Notice that other components (κ₁ and α₁) are computed in the previous iteration.
The following constants are defined:

$$\begin{matrix}C = \frac{1}{{I}_{1} + {I}_{2}} & ( 7 ) \\ {C}_{s} = \frac{\overline{κ} sin \overline{α}}{C} & ( 8 ) \\ {C}_{c} = \frac{\overline{κ} cos \overline{α}}{C} & ( 9 ) \\ {B}_{s} = {κ}_{1} \cdot {I}_{1} \cdot sin ( {α}_{1} ) & ( 10 ) \\ {B}_{c} = {κ}_{1} \cdot {I}_{1} \cdot cos ( {α}_{1} ) & ( 11 )\end{matrix}$$

Then, the system from Eq. (6) becomes:

Cₛ=Bₛ+κ₂·I₂·sin α₂ (12)

C_{c}=B_{c}+κ₂·I₂·*cos α₂ (13)
Therefore:

$$\begin{matrix}{α}_{2} = a tan 2 ( {C}_{s} - {B}_{s} , {C}_{c} - {B}_{c} ) , \\ and & ( 14 ) \\ {κ}_{2} = \{ \begin{matrix}\frac{{C}_{c} - {B}_{c}}{{I}_{2} cos {α}_{2}} & cos {α}_{2} \neq 0 \\ \frac{{C}_{s} - {B}_{s}}{{I}_{2} sin {α}_{2}} & sin {α}_{2} \neq 0\end{matrix} & ( 15 )\end{matrix}$$

The full model can be computed iteratively: In the first step, κ₁ is and α₁ are computed from κ₀ and α₀, in the second step κ₂ and α₂ are computed from κ₁ and αᵢ, . . . , finally κₙ₋₁ and αₙ₋₁ are computed from Kₙ₋₂ and aₙ₋₂.

The computed curvatures κᵢ and rotation angles α, can be used to assemble the active cannula configuration per WO 2007/042986. The compensation effected by the above calculation improves conformity of behavior of the deployed active cannula device with the planned path. The planned path having been calculated in turn to conform to body tissues.

Then, at 504 a corrected set of cannulas with defined curvatures and orientations is produced. This corrected set of cannulas will be produced responsive to an output specification resulting from the correction 503. The outputs specification will preferably include:
an ordered sequence of numbered cannulas;
a respective curvature for each cannula;
a respective length for each cannula; and
a respective orientation of each cannula.

FIGS. 7A and B show deployed nested cannulas κ̅₀̅, κ̅₁̅, κ̅₂̅, . . . , κ̅ₙ̅₋̅₁̅ in accordance with the invention, in which several different curvatures and several elasticities are illustrated. None of the tubes is straight. Generally, the smaller cannulas will have larger maximal curvature and therefore have the possibility of smaller radii of curvature than the larger cannulas. Due to the fact that this is a planar drawing, the deployed device is shown within

a plane. In reality, the deployed device will have a three dimensional shape, in which various curvatures are in different planes.

In the next pararaphs, some alternate embodiments are proposed to simplify the model. Listing some examples of alternate embodiments is not intended to limit the application—as the skilled artisan might come up with other alternatives to simplify calculations.

Modification 1

If the planned path includes alternating straight-curve segments, interaction can be precomputed pairwise for any combination of N planned segments. FIG. 7B shows a set of concentric cannulas in a deployed state with alternating straight and curved segments.

Segments that are straight are shown with net curvature equal zero, e.g. κ̅₁̅, κ̅₃̅ and segments that are curved are shown at κ̅₀̅, κ̅₂̅, κ̅₄̅. This calculation advantage makes it possible to define a set of N prefabricated tubes that can be used to assemble any planned path consisting of M<=N tubes. In general, having a set of tubes that has a discrete set of curvatures for each tube diameter from which to select elements of the final assembly will simplify calculation.

This modification simplifies premanufacturing of tubes per U.S. application Ser. No. 12/088,870 of Trovato et al.

### Modification 2

To improve modification 1, tubes can be selected to have balanced moments of inertia—Iᵢ=I for every i. In this case, if two curved tubes compensate for each other to yield a straight segment they have the same curvature—with opposite orientations. In such cases, the number of prefabricated tubes could be reduced by factor 2, as compared to Modification 1 alone.

### Modification 3

There are a number of properties that might affect net curvature. These include angular rotation, tube radius of curvature, modulus of elasticity (elasticity modulus), and moment of inertial. While it may be that all each tube in an assembly of concentric cannulas may have a distinct value for each of these properties, calculation or manufacturing may be simplified by having all tubes share at least one of the properties.

Generally, there may be manufacturing advantages to having device tube(s) include alternating sequences of straight and curved segments. Tubes may have more than one curvature along their length, as shown in FIG. 8. This tube has a portion with curvature k2=0 and portion having a radius of curvature r1—and curvature k1. Such a tube might be treated as two tubes in the calculations above, with each tube having the same moment of inertia and Young's modulus, but different curvatures. Also, the calculation has to be adjusted to show that these “two” tubes do not interact with each other.

Realistically, in an assembly with more than about three tubes, the innermost tubes will stop having a significant effect on the total curvature of the device in areas where there is overlap. Calculation may be simplified by applying a threshold to determine how many tubes are considered to contribute to a net curvature. One type of threshold might relate to determining when an inner tube has a moment of inertia that is less than some predetermined threshold percentage of the moment of inertia of some outer tube. One such threshold percentage might be 10%. Another threshold might be to consider, in region of overlap, only a predetermined number of outer tubes, such as three.

The result of the preceding calculations should be a tube specification, typically in the form of a list of tubes with sequence numbers. Each sequence number will be correlated with a diameter of the tube and accompanied by tube specifications such as curvature, length, and orientation. The output may be in the form of an animation or some other graphic output. FIG. 11 shows such an animation, where the sequential frames illustrate a concentric cannula advancing in the lung. Such an animation may be accompanied by audio or text instructions relating to tube size or deployment of the tubes. A manufacturer, upon receiving the specification, will produce a device including a set of concentric cannulas. The cannulas will preferably be shipped in an airtight, sterile packaging arranged with their distal ends flush. Deployment will preferably be by inserting the assembly and then advancing the inner tubes in reverse order of tube diameter, until the assembly has all the proximal ends of the tubes flush. Other orders are possible and might entail different types of tube interactions.

Manufacturers of such cannulas will likely be making many assemblies at a time using automated processes responsive to multiple individual requests from multiple medical providers. FIG. 10 shows schematically a number of individual examination sites 1001 providing examination data over the internet 1002 to an assembler of sets of cannulas 1003, which in turn ships many assembled sets of concentric cannulas 1004 to appropriate clinics and hospitals where they can be deployed into patients.

Generally, planning concentric cannula devices may start with a discrete set of pre-ordered and stored tubes 1005. This discrete set reduces manufacturing costs by reducing the number of tubes, especially the number of specific curvatures a manufacturer has to have in stock. The method in accordance with the invention allows for a more varied set of tube curvatures to be used, since the type of tube needed to make an adjustment in accordance with the calculations performed above would only be requested at 1006 after calculations are performed. Preferably, customized tube orders could be minimized by starting from a concentric cannula device composed of tubes selected from a discrete set; then performing an adjustment calculation as described above; and finally only ordering a custom tube when calculation reveals a need for adjustment that differs from the starting device by an amount that exceeds some threshold. A set of concentric cannula devices produced in accordance with the invention will accordingly normally have a greater diversity of component tubes than might be expected in the prior art. Alternatively, modification 1 will allow discrete set of pre-ordered and stored tubes.

From reading the present disclosure, other modifications will be apparent to persons skilled in the art. Such modifications may involve other features which are already known in the design, manufacture and use of medical robotics and which may be used instead of or in addition to features already described herein. Although claims have been formulated in this application to particular combinations of features, it should be understood that the scope of the disclosure of the present application also includes any novel feature or novel combination of features disclosed herein either explicitly or implicitly or any generalization thereof, whether or not it mitigates any or all of the same technical problems as does the present invention. The applicants hereby give notice that new claims may be formulated to such features during the prosecution of the present application or any further application derived therefrom.

The word “comprising”, “comprise”, or “comprises” as used herein should not be viewed as excluding additional elements. The singular article “a” or “an” as used herein should not be viewed as excluding a plurality of elements. The word “or” should be construed as an inclusive or, in other words as “and/or”.

### Bibliography:

DE 4223897 C2
U.S. Pat. No. 6,572,593
R. J. Webster & N. J. Cowan, “Toward Active Cannulas: Miniature Snake-like Surgical Robots” 2006 IEEE/RSJ (October 2006, Beijing, China) pp. 2857-2863
K. I. Trovato, A* Planning in Discrete Configuration Spaces of Autonomous Systems, (U. of Amsterdam 1996)
U.S. Pat. No. 6,251,115
P Sears et al., “Inverse kinematics of concentric tube steerable needles”, IEEE Conf. on Robotics and Automations, pp. 1887-1892 (2007)

1. A computer method for planning a configuration of a device, the method comprising executing operations on a data processing apparatus, the operations comprising:
receiving, as an input, path information for the device, the path information including at least one net curvature specification for a set of concentric cannulas including an initial indication of which cannulas are in the set of concentric cannulas;
determining at least one actual net curvature, responsive to calculating interactions between cannulas in the initial indication, the interactions being due to at least one curvature affecting property of the cannulas; and
creating an output specification for the set of concentric cannulas, responsive to the determining

2. The method of claim 1, wherein the curvature affecting property comprises elasticity.

3. The method of claim 1, wherein the curvature affecting property comprises radius of curvature.

4. The method of claim 1, wherein the curvature affecting property comprises moment of inertia.

5. The method of claim 1, wherein the curvature affecting property comprises more than one property from amongst a group including elasticity, radius of curvature, and moment of inertia.

6. The method of claim 1, wherein creating comprises iteratively accumulating interaction information while considering cannulas in order from an innermost cannula to an outermost cannula based on the initial indication.

7. The method of claim 1, wherein the output specification includes:
an ordered sequence of numbered cannulas;
a respective curvature for each cannula;
a respective length for each cannula; and
a respective orientation of each cannula.

8. The method of claim 1, wherein the operations further comprise:
receiving a representation of a space to be explored in the form of a configuration space data structure embodied on at least one medium;
receiving a representation of a set of possible tubes in the form of a neighborhood data structure embodied on at least one medium; and
calculating the path information based on applying A* to propagate cost waves through the configuration space using a metric and the neighborhood to yield the initial indication.

9. The method of claim 1, wherein creating the output specification comprises within the initial indication, starting from the most distal cannula;
calculating an interaction between a current cannula and at least one more proximal cannula to yield the actual net curvature of the current and more proximal cannulas;
if the actual net curvature of the cannulas in the calculating operation differs from the net curvature specifications, changing a specification of the current cannula and/or the more proximal cannula; and
iterating the calculating and changing operations until a most proximal cannula specification is reached.

10. The method of claim 1, wherein the path information comprises alternating straight and curved segments.

11. The method of claim 10, wherein straight segments are achieved using tubes with balanced curvature affecting properties.

12. The method of claim 1, wherein all cannulas in the output set have at least one same property from a group comprising: moment of inertia, elasticity, and curvature.

13. An assembly of concentric cannulas, assembled responsive to the output specification of the method of claim 1, and ready for deployment in accordance with the path information.

14. A plurality of assemblies of concentric cannulas in accordance with claim 13.

15. The assemblies of claim 14, wherein at least one value, which is associated with a property of at least one of the cannulas from the initial indication, is changed in creating the output specification and after determining the actual net curvature.

16. The method of claim 1, wherein the output specification is in the form of an animation.

17. An assembly of concentric tubes, the tubes being adapted to be deployed by extension to follow a planned path, the assembly comprising at least first and second tubes having complementary values of a curvature affecting property such that, in an area of overlap, the first and second tubes interact through mutually opposing forces to achieve a desired curvature to follow the planned path, the desired curvature being different from respective curvatures of both the first and second tubes.

18. The assembly of claim 17, wherein the curvature affecting property is radius of curvature.

19. The assembly of claim 17, wherein the curvature affecting property is elasticity.

20. The assembly of claim 17, wherein the curvature affecting property is moment of inertia.

21. The assembly of claim 17, wherein the planned path comprises alternating straight and curved segments and the first and second tube interact to achieve a straight segment.

22. The assembly of claim 17, wherein at least some tubes in the assembly are selected from a set including a discrete set of curvatures for each tube diameter.

23. The assembly of claim 22, wherein at least one tube is altered from the discrete set, responsive to the values of the curvature affecting property, so that the assembly follows the planned path.

24. A computer readable medium embodying program code for causing a data processing apparatus to perform operations, the operations comprising:
receiving, as an input, path information for the device, the path information including at least one net curvature specification for a set of concentric cannulas including an initial indication of which cannulas are in the set of concentric cannulas;
determining at least one actual net curvature, responsive to calculating interactions between cannulas in the initial indication, the interactions being due to at least one curvature affecting property of the cannulas; and
creating an output specification for the set of concentric cannulas, responsive to the determining.
